(19) United States
(12) Patent Application Publication
(10) Pub. No.: US 2005/0202020 A1
(43) Pub. Date: Sep. 15, 2005

(54) Diagnosing and treating cancer

(76) Inventors: Jeffrey Ross, Lebanon Springs, NY (US); Karen Gray, Annandale, NJ (US)

Correspondence Address: FISH & RICHARDSON PC, 225 FRANKLIN ST, BOSTON, MA, 02110, US

(21) Appl. No.: 11/027,954

(22) Filed: Dec. 30, 2004

Related U.S. Application Data
(60) Provisional application No. 60/535,260, filed on Jan. 9, 2004.

(51) Int. Cl. A61K 39/395

(52) U.S. Cl. 424/155.1; 424/178.1

(57) ABSTRACT

Methods for treating or diagnosing cancers of the female reproductive tract and childhood cancers are disclosed. The methods described herein use binding agents, e.g., antibodies, specific for the extracellular domain of human prostate specific membrane antigen (PSMA).

### RELATED APPLICATIONS

This application claims the benefit of U.S. Provisional Application No. 60/535,260 filed Jan. 9, 2004, the entire contents of which are hereby incorporated by reference herein.

### FIELD OF THE INVENTION

The invention relates to the use of a binding agent specific for prostate specific membrane antigen (PSMA) to diagnose, treat or prevent a non-prostate cancer, e.g., a cancer of the female reproductive tract such as ovarian, cervical, endometrial, gestational trophoblastic, uterine, vaginal, vulvar or pelvic cancers, or a childhood cancer, e.g., Wilm's tumors or neuroblastoma.

### BACKGROUND OF THE INVENTION

Prostate specific membrane antigen (PSMA) is transmembrane folate hydrolase with restricted expression limited to normal prostate tissue, prostate cancer and the neovasculature of various non-prostate cancers. Non-prostate cancer expression of PSMA is currently being pursued as a target for diagnostic imaging and anti-cancer antibody therapeutics.

### SUMMARY OF THE INVENTION

The invention is based, in part, on the discovery that prostate specific membrane antigen (PSMA) is associated with cancer of the female reproductive system (e.g., ovarian cancers), childhood cancers (e.g., Wilm's tumors and neuroblastomas) as well as other adult and childhood cancers (e.g., renal, breast, lung and colon cancers). Enhanced expression of PSMA protein was detected within endothelial cells of neovasculature associated with cancerous cells of the female reproductive system, with cancerous cells of various childhood cancers and with cancerous cells of various cancers affecting adults and children.

Accordingly, in some aspects, the invention provides methods and compositions for diagnosing, treating or preventing cancers of the female reproductive system, including ovarian, cervical, endometrial, gestational trophoblastic, uterine, vaginal, vulvar or pelvic cancers, using binding agents, e.g., antibodies or antigen binding fragments thereof specific for PSMA, e.g., specific for the extracellular region of PSMA. In a preferred embodiment, a cancer of the female reproductive system can be a cancer in which the primary lesion originates in a tissue of the female reproductive system. In other preferred embodiments, a cancer of the female reproductive system can be one in which the cancer originates in, or wherein the primary lesion is in, a tissue other than a tissue of the female reproductive system. Thus, in such embodiments, a cancer that originates outside a tissue of the female reproductive system but that metastasizes or otherwise migrates into a tissue of the female reproductive system, is treated, e.g., a lesion that originates in a cancer of the lung, colon or breast.

In other aspects, the invention features methods and compositions for diagnosing, treating or preventing cancer in a child, e.g., childhood cancers such as childhood cancers of the kidney (e.g., Wilm's tumor) or nervous system (e.g., neuroblastoma).

In yet other aspects, the invention provides methods and compositions for diagnosing, treating and preventing other cancers affecting adults and children.

In one aspect, the invention features a method of treating, e.g., ablating or killing, a cell, e.g., a cancerous cell (e.g., an aberrant PSMA-expressing cell). The methods include contacting the cell, or a nearby cell, e.g., a vascular endothelial cell proximate to the cell, with a binding agent, e.g., an antibody or antigen-binding fragment thereof, that specifically binds PSMA, in an amount sufficient to treat, e.g., ablate or kill, the cell, or nearby cell. Methods of the invention can be used, for example, to treat or prevent a disorder, e.g., a cancer of the female reproductive system, a childhood cancer, or other cancer described herein, by administering to a subject a PSMA-binding agent, e.g., an anti-PSMA antibody or antigen-binding fragment thereof, in an amount effective to treat or prevent such disorder.

In some embodiments, the methods described herein can be used on cells in culture, e.g., in vitro or ex vivo. For example, cells derived from neovasculature of cancerous tissue associated with a cancer of the female reproductive system, a childhood cancer, or other cancer described herein can be cultured in vitro in culture medium, and the contacting step can be effected by adding the PSMA-binding agent to the culture medium. In some embodiments, the methods described herein can be performed on cells (e.g., cancerous cells) present in a subject, e.g., cells present in the cancerous tissue or neovasculature of cancerous tissue as part of an in vivo (e.g., therapeutic or prophylactic) protocol. For in vivo embodiments, the contacting step is effected in a subject and typically includes administering the binding agent to the subject under conditions effective to permit both binding of the binding agent to the cell, or a vascular endothelial cell proximate to the cell, and the treating, e.g., the killing or ablation, of the cell or the proximate cell.

The methods described herein can be used to treat or prevent a cancer of the female reproductive system, a childhood cancer or other cancer affecting adults or children.

Thus, in one aspect, the invention provides a method of treating a subject having a cancer selected from the group consisting of a cancer of the female reproductive tract, a childhood cancer, or other cancer described herein. The method includes administering to the subject an effective amount of a PSMA binding agent, e.g., an anti-PSMA antibody or antigen binding fragment thereof that binds to the extracellular domain of PSMA, thereby treating the cancer.

In some embodiments, the cancer is a cancer of the female reproductive tract, e.g., a cancer selected from the group consisting of ovarian, cervical, endometrial, uterine, vaginal, vulvar or pelvic cancers and gestational trophoblastic tumors. In some embodiments, the female reproductive tract cancer is ovarian cancer. In some embodiments, the cancer is a childhood cancer, e.g., a childhood cancer selected from the group consisting of neuroblastomas, brain cancers, lymphomas, Wilm's tumors, bone cancers, endocrine cancers, primitive neuroectodermal tumor, retinoblastomas, rhabdomyosarcomas, and ovarian germ cell tumors. In some embodiments, the childhood cancer is Wilm's tumor. In some embodiments, the childhood cancer is neuroblastoma. In other embodiments, the cancer is renal cancer (e.g., a clear cell renal cancer, chromophilic renal cancer, chromophobic renal cancer, oncocytic cancer or collecting duct or Bellini duct cancer). In another embodiment, the cancer is breast cancer (e.g., infiltrating ductal breast cancer (e.g., mucinous, medullary, papillary or tubular), infiltrating labular carcinoma or sarcoma). In yet another embodiment, the cancer is lung cancer (e.g., small cell lung cancer, non-small cell lung cancer or bronchial gland cancer).

As used herein, “cancers of the female reproductive system” refers to cancers present in one or more tissues or organs of the female reproductive system. Examples of cancers of the female reproductive system include ovarian cancers, e.g., ovarian epithelial cancers (e.g., adenocarcinoma, adenofibroma, Brenner tumor, clear cell carcinoma, clear cell tumor, cystadenocarcinoma, endometrioid carcinoma, mucinous cystadenocarcinoma, mucinous cystadenoma mucinous tumor, serous cystadenocarcinoma, serous cystadenoma, serous tumor, clear cell carcinoma, and borderline ovarian tumors), ovarian germ cell cancers (e.g., dysgerminoma and nondysgerminoma, e.g., endodermal sinus tumor, embryonal carcinoma, polyembryoma, choriocarcinoma, papillary serous adenocarcinoma, and teratoma), ovarian sex cord-stromal cell cancers (e.g., Sertoli-Leydig cell tumor, granulosa-theca cell tumor, theca cell tumor, thecoma, fibroma, and gonadoblastoma), cervical cancers (e.g., squamous cell carcinoma), endometrial cancers, uterine cancers (e.g., actinomycosis, adenocarcinoma, papillary adenocarcinoma, clear cell adenocarcinoma of the endocervix, clear cell adenocarcinoma, polyp, stromal sarcoma, leiomyoma, malignant mixed Mullerian tumor, mixed mesodermal tumor, homologous type, and myometrial hypertrophy), vaginal cancers, vulvar cancers, pelvic cancers and gestational trophoblastic tumors. In a preferred embodiment, a cancer of the female reproductive system can be a cancer in which the primary lesion originates in a tissue of the female reproductive system. In other preferred embodiments, a cancer of the female reproductive system can be one in which the cancer originates in, or wherein the primary lesion is in, a tissue other than a tissue of the female reproductive system. Thus, in such embodiments, a cancer that originates outside a tissue of the female reproductive system but that metastasizes or otherwise migrates into a tissue of the female reproductive system, is treated, e.g., a lesion that originates in a cancer of the lung, colon or breast.

Various stages of a cancer, e.g., ovarian cancer, can be treated or diagnosed. Cancer that originates in the female reproductive tract can be treated prior to spreading to other areas or after metastasizing to other areas. Ovarian cancer is illustrative. Stages of ovarian cancer include: stage 1 (e.g., ovarian cancer in one or both of the ovaries); stage 2 (e.g., cancer in one or both of the ovaries and in the uterus and/or fallopian tubes); stage 3 (e.g., cancer in one or more of the ovaries and one or more organs in the abdominal cavity (e.g., abdominal lymph nodes, liver or bowel)); stage 4 (e.g., cancer has metastasized to outside the abdominal cavity, e.g., to one or more of the liver, lung, brain and lymph nodes of the neck). The invention can also be used to diagnose or treat recurrent cancer of the female reproductive system, e.g., recurrent ovarian cancer.

As used herein, “childhood cancers” refers to cancers that principally affect children. Childhood cancers include cancers of the sympathetic nervous system, e.g., neuroblastomas, brain cancers (e.g., meduloblastoma and malignant glioma (e.g., astrocytomas and glioblastomas)), primative neuroectodermal tumors, lymphomas, renal cancer, e.g., Wilm's tumors, bone cancers, retinoblastomas, endocrine cancers (e.g., thyroid cancer and adrenal gland cancer), rhabdomyosarcomas, and ovarian germ cell tumors. Additional information regarding these cancers, including existing methods of diagnosis, evaluation, and treatment, can be obtained from the National Cancer Institute of the National Institutes of Health, e.g., on the world wide web at cancer.gov/cancerinformation/cancertype/childhood, or at the National Childhood Cancer Foundation (NCCF) website, on the world wide web at nccf.org/childhoodcancer. Methods of the invention also include the treatment of any cancer in a child, e.g., in an infant (e.g., a child less than 1 year old), a child more than 1 year old, a child less than 5 years old, a prepubescent child, or a postpubescent child.

Wilm's tumor is a cancerous tumor of the kidney, also referred to as nephroblastoma. It is the most common form of kidney cancer in children, and usually occurs in children younger than 5 years old. Various stages of Wilm's tumors can be diagnosed and treated. Stages of Wilm's tumor's include: stage 1 (cancer is found only in the kidney); stage 2 (cancer is found in the kidney and areas near the kidney such as to fat, soft tissue, blood vessels and/or the renal sinus); stage 3 (cancer is found in kidney and in areas near the kidney including organs or vessels near the kidney, the abdomen and/or lymph nodes near the kidney); stage 4 (cancer has spread to organs further away from the kidney such as lung, liver, bone and brain); and stage V (cancer is found in both kidneys). Within each of the stages of Wilm's tumor, the tumor can be categorized as favorable or unfavorable based upon the histology. The presence of anaplasia, clear cell sarcoma and/or rhabdoid tumor is associated with an unfavorable categorization. The invention can be used to treat or diagnose recurrent Wilm's tumors.

Neuroblastoma, a cancer of the sympathetic nervous system, is predominantly a tumor of early childhood, with two thirds of the cases presenting in children younger than 5 years of age. Various stages of neuroblastoma can be diagnosed or treated. Stages of neuroblastomas include: stage 1 (tumor is confined to its area of origin and lymph nodes near the tumor do not contain the cancer); stage 2A (tumor is confined to one side of the body but cannot be removed completely; lymph nodes near the tumor do not contain cancer); stage 2B (tumor is confined to one side of the body and lymph nodes on the same side of the body show evidence of cancer; lymph nodes on opposite side of the body do not show evidence of cancer); stage 3 (tumor crosses over the midline of the body and lymph nodes may or may not be affected; tumor is on one side of the body and lymph nodes on the opposite side of the body are affected); stage 4 (cancer is spread to other organs and tissues such as lymph nodes, bone, bone marrow, liver, etc.); and stage 4S (tumor is confined to its area of origin with limited spread to the liver, skin and/or bone marrow). The invention can also be used to treat or diagnose recurrent neuroblastomas.

Other cancers described herein are cancers that can affect adults or children. These cancers include renal cancer (e.g., a clear cell cancer, chromophilic cancer, chromophobic cancer, oncocytic cancer or collecting duct or Bellini duct cancer); breast cancer (e.g., infiltrating ductal breast cancer (e.g., mucinous, medullary, papillary or tubular), infiltrating labular carcinoma or sarcoma) and lung cancer (e.g., small cell lung cancer, non-small cell lung cancer or bronchial gland cancer).

In some embodiments, the binding agent used in the methods and compositions of the invention, interacts with, e.g., binds to, PSMA, preferably human PSMA, with high affinity and specificity. For example, the binding agent binds to human PSMA with an affinity constant of at least 10⁷ M⁻¹, preferably, between 10⁸ M⁻¹ and 10¹⁰ M⁻¹, or about 10⁹ M⁻¹. Typically, the binding agent binds to the extracellular domain of PSMA, e.g., the extracellular domain of human PSMA (e.g., amino acids 44-750 of human PSMA).

The binding agent can be an antibody (e.g., a monospecific, or a recombinant or modified antibody) or an antigen-binding fragment thereof, a small molecule, or a PSMA ligand. Examples of ligands and small molecules that can be used in the invention include those described in PCT Publication No.: WO 01/74845, PCT Publication No.: WO 02/098885 and PCT Publication No.: WO 03/060523, the contents of all of which are incorporated herein by reference. In some embodiments, the antibodies are those having one or more complementarity determining regions (CDRs) from a J591, J415, J533 or E99 antibody or from an antibody which competes with or has an overlapping epitope with one of these antibodies. For example, the antibody has a light chain variable region comprising one or more complementarity determining regions (CDRs) from a monoclonal antibody selected from the group consisting of J591, J415, J533 and E99 or from an antibody which competes with or has an overlapping epitope with one of these antibodies, and/or a heavy chain variable region comprising one or more CDRs from a monoclonal antibody selected from the group consisting of J591, J415, J533 and E99 or from an antibody which competes with or has an overlapping epitope with one of these antibodies. In some embodiments, the antibody or antigen binding portion thereof comprises all six CDRs from murine J591, or all six CDRs from murine J415. In other embodiments, the antibodies are those having one or more complementarity determining regions (CDRs) from a 4A3, 7F12, 8A11, 8C12, 16F9 026 or PSMA 4.40 antibody or from an antibody which competes with or has an overlapping epitope with one of these antibodies, e.g., having a light chain variable region comprising one or more complementarity determining regions (CDRs) from a monoclonal antibody selected from the group consisting of 4A3, 7F12, 8A11, 8C12, 16F9 026 and PSMA 4.40 or from an antibody which competes with or has an overlapping epitope with one of these antibodies, and/or a heavy chain variable region comprising one or more CDRs from a monoclonal antibody selected from the group consisting of 4A3, 7F12, 8A11, 8C12, 16F9 026 and PSMA 4.40 or from an antibody which competes with or has an overlapping epitope with one of these antibodies. In some embodiments, the antibody or antigen binding portion thereof comprises all six CDRs from one of the aforementioned antibodies.

In some embodiments, the binding agent is an anti-PSMA monospecific antibody (e.g., a monoclonal, chimeric, CDR-grafted, humanized, e.g., a humanized mouse antibody, deimmunized, e.g., a deimmunized mouse antibody, or human antibody) or an antigen-binding fragment thereof. The anti-PSMA antibodies (e.g., recombinant or modified antibodies) can be full-length (e.g., an IgG (e.g., an IgG1, IgG2, IgG3, IgG4), IgM, IgA (e.g., IgA1, IgA2), IgD, and IgE, but preferably an IgG) or can include only an antigen-binding fragment (e.g., a Fab, F(ab′)₂ or scFv fragment, or one or more CDRs). An antibody, or antigen-binding fragment thereof, can include two heavy chain immunoglobulins and two light chain immunoglobulins, or can be a single chain antibody. The antibodies can, optionally, include a constant region chosen from a kappa, lambda, alpha, gamma, delta, epsilon or a mu constant region gene. A preferred anti-PSMA antibody includes a heavy and/or light chain constant region substantially from a human antibody, e.g., a human IgG1 constant region or a portion thereof. In some embodiments, the anti-PSMA antibodies are human antibodies.

The antibody (or fragment thereof) can be a murine or a human antibody. Examples of murine monoclonal antibodies that can be used include a E99, J415, J533 and J591 antibody, which are produced by hybridoma cell lines having an ATCC Accession Number HB-12101, HB-12109, HB-12127, and HB-12126, respectively. Also within the scope of the invention are methods and compositions using antibodies, or antigen-binding fragments thereof, which bind overlapping epitopes of, or competitively inhibit, the binding of an anti-PSMA antibody disclosed herein to PSMA, e.g., antibodies which bind overlapping epitopes of, or competitively inhibit, the binding of one or more of monoclonal antibody E99, J415, J533, J591, 4A3, 7F12, 8A11, 8C12, 16F9 026 or PSMA 4.40 to PSMA. Any combination of anti-PSMA antibodies can be used, e.g., two or more antibodies that bind to different regions of PSMA, e.g., antibodies that bind to two different epitopes on the extracellular domain of PSMA.

In some embodiments, the binding agent is an anti-PSMA antibody that binds to all or part of the epitope of an antibody described herein, e.g., a J591, E99, J415, J533, 4A3, 7F12, 8A11, 8C12, 16F9 026 and PSMA 4.40 antibody. The anti-PSMA antibody can inhibit, e.g., competitively inhibit, the binding of an antibody described herein, e.g., a J591, E99, J415, J533, 4A3, 7F12, 8A11, 8C12, 16F9 026 and PSMA 4.40 antibody, to human PSMA. An anti-PSMA antibody may bind to an epitope, e.g., a conformational or a linear epitope, which epitope when bound prevents binding of an antibody described herein, a J591, E99, J415, J533, 4A3, 7F12, 8A11, 8C12, 16F9 026 and PSMA 4.40 antibody. The epitope can be in close proximity spatially or functionally associated, e.g., an overlapping or adjacent epitope in linear sequence or conformationally to the one recognized by the J591, E99, J415, J533, 4A3, 7F12, 8A11, 8C12, 16F9 026 or PSMA 4.40 antibody.

In one embodiment, the anti-PSMA antibody binds to an epitope located wholly or partially within the region of about amino acids 120 to 500, e.g., 130 to 450, 134 to 437, or 153 to 347, of human PSMA. Typically, the epitope includes at least one glycosylation site, e.g., at least one N-linked glycosylation site (e.g., the N-linked glycosylation site located at about amino acids 190-200, preferably at about amino acid 195, of human PSMA).

In other embodiments, the antibody (or antigen-binding fragment thereof) is a recombinant or modified anti-PSMA antibody chosen from, e.g., a chimeric, a CDR-grafted, a humanized, a deimmunized, or an in vitro generated antibody (or an antigen-binding fragment thereof). As discussed herein, the modified antibodies can be CDR-grafted, humanized, deimmunized, or more generally, antibodies having CDRs from a non-human antibody, e.g., murine J591, J415, J533 or E99 antibody and a framework that is selected as less immunogenic in humans, e.g., less antigenic than the murine framework in which a murine CDR naturally occurs. In one embodiment, a modified antibody is a deimmunized anti-PSMA antibody, e.g., a deimmunized form of E99, J415, J533 or J591 (e.g., a deimmunized form of an antibody produced by a hybridoma cell line having an ATCC Accession Number HB-12101, HB-12109, HB-12127 and HB-12126, respectively). Typically, the antibody is a deimmunized form of J591 or J415 (referred to herein as “deJ591” or “deJ415” respectively). Most preferably, the antibody is a deimmunized form of J591. The antibody can be a human antibody, e.g., a human antibody made in a non-human animal, e.g., a mouse.

The binding agent, e.g., the anti-PSMA antibody, or antigen-binding fragment thereof, described herein can be used alone, e.g., can be administered to a subject or used in vitro, in non-derivatized or unconjugated forms. For example, the unconjugated form of an anti-PSMA antibody can ablate or kill the PSMA-expressing cell by antibody dependent-cell killing mechanisms such as complement mediated cell lysis and/or effector cell-mediated cell killing. Typically, the anti-PSMA antibody binds to the cell surface of the cell that expresses PSMA (e.g., a vascular endothelial cell associated with a cancerous tissue), and, in particular, to the cell surface of living cells.

In some embodiments, the binding agent, e.g., an anti-PSMA antibody or fragment thereof, is also internalized with PSMA, which permits intercellular delivery of a molecular entity conjugated to the antibody. The binding agent, e.g., an anti-PSMA antibody, or antigen-binding fragment thereof, can be derivatized or linked (coupled) to another molecular entity, typically a label or a therapeutic (e.g., a cytotoxic or cytostatic) moiety or agent. The molecular entity can be, e.g., another peptide, protein, a non-peptide chemical compound, isotope, etc. The anti-PSMA antibody, or antigen-binding fragment thereof, can be functionally linked, e.g., by chemical coupling, genetic fusion, non-covalent association or otherwise, to one or more other molecular entities. For example, the anti-PSMA antibody, or antigen-binding fragment thereof, can be coupled to a label, such as a fluorescent label, a biologically active enzyme label, a radioisotope (e.g., a radioactive ion), a nuclear magnetic resonance active label, a luminescent label, or a chromophore. In other embodiments, the anti-PSMA antibody, or antigen-binding fragment thereof, can be coupled to a therapeutic agent, e.g., a cytotoxic moiety, e.g., a therapeutic drug, a radioisotope, molecules of plant, fungal, or bacterial origin, or biological proteins (e.g., protein toxins), or mixtures thereof.

Thus, the antibodies of the present invention can be used to deliver a variety of therapeutic agents, e.g., a cytotoxic moiety, e.g., a therapeutic drug, a radioisotope, molecules of plant, fungal, or bacterial origin, or biological proteins (e.g., protein toxins) or particles (e.g., a recombinant viral particle, e.g., via a viral coat protein), or mixtures thereof. The therapeutic agent can be an intracellularly active drug or other agent, such as short-range radiation emitters, including, for example, short-range, high-energy α-emitters, as described herein. In some preferred embodiments, the anti-PSMA antibody, or antigen binding fragment thereof, can be coupled to a molecule of plant or bacterial origin (or derivative thereof), e.g., a maytansinoid (e.g., maytansinol or the DM1 maytansinoid). DM1 is a sulfhydryl-containing derivative of maytansine that can be linked to antibodies via a linker, e.g., a disulfide linker that releases DM1 when inside target cells. Maytansine is a cytotoxic agent that causes cell death by preventing the formation of microtubules and depolymerization of extant microtubules. It is 100- to 1000-fold more cytotoxic than anticancer agents such as doxorubicin, methotrexate, and vinca alkyloid, which are currently in clinical use. Alternatively, the anti-PSMA antibody, or antigen binding fragment thereof, can be coupled to a taxane, a calicheamicin, a proteosome inhibitor, or a topoisomerase inhibitor. [(1R)-3-methyl-1-[[(2S)-1-oxo-3-phenyl-2-[(3-mercaptoacetyl) amino]propyl]amino]butyl]-Boronic acid is a suitable proteosome inhibitor. N,N′-bis[2-(9-methylphenazine-1-carboxamido)ethyl]-1,2-ethanediamine is a suitable topoisomerase inhibitor. Enzymatically active toxins and fragments thereof are exemplified by diphtheria toxin A fragment, nonbinding active fragments of diphtheria toxin, exotoxin A (from Pseudomonas aeruginosa), ricin A chain, abrin A chain, modeccin A chain, α-sacrin, certain Aleurites fordii proteins, certain Dianthin proteins, Phytolacca americana proteins (PAP, PAPII and PAP-S), Morodica charantia inhibitor, curcin, crotin, Saponaria officinalis inhibitor, gelonin, mitogillin, restrictocin, phenomycin, and enomycin. In some embodiments, the anti-PSMA antibody is conjugated to a maytansinoid, e.g., maytansinol (see U.S. Pat. No. 5,208,020), CC-1065 (see U.S. Pat. Nos. 5,475,092, 5,585,499, 5,846,545). Procedures for preparing enzymatically active polypeptides of the immunotoxins are described in W084/03508 and W085/03508, which are hereby incorporated by reference, and in the appended Examples below. Examples of cytotoxic moieties that can be conjugated to the antibodies include adriamycin, chlorambucil, daunomycin, methotrexate, neocarzinostatin, and platinum.

A compound emitting radiation, e.g., a radioisotope, can be an α-, β-, or γ-emitter, or a β- and γ-emitter. Radioisotopes useful as therapeutic agents include yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), and rhodium (¹⁸⁸Rh). Radioisotopes useful as labels, e.g., for use in diagnostics, include iodine (¹³¹I or ¹²⁵I), indium (¹¹¹In), technetium (⁹⁹mTc), phosphorus (³²P), carbon (¹⁴C), and tritium (³H), or one of the therapeutic isotopes listed above. The anti-PSMA antibody, or antigen-binding fragment thereof can also be linked to another antibody to form, e.g., a bispecific or a multispecific antibody.

The subject can be mammal, e.g., a primate, e.g., a higher primate, e.g., a human (e.g., a patient having, or at risk of, a disorder described herein, e.g., a cancer of the female reproductive tract or a childhood cancer as described herein). In some embodiments, the subject is a human patient having ovarian cancer, Wilm's tumors, or neuroblastoma.

The PSMA binding agent, e.g., a PSMA binding agent as described herein, is typically administered to the subject systemically (e.g., intravenously, intramuscularly, by infusion, e.g., using an infusion device, subcutaneously, transdermally, or by inhalation). In those embodiments where the PSMA binding agent is a small molecule, it can be administered orally. In some embodiments, the PSMA binding agent is administered locally to an affected area, e.g., the neovasculature of a cancerous tissue, i.e., the neovasculature of a tumor.

The methods described herein can further include the step of monitoring the subject, e.g., for a reduction in one or more of: a reduction in size, growth rate, etc. of a cancerous tissue, i.e., a tumor; a reduction in the subject's symptoms; reduced number of proliferating cells, or any other parameter related to improvement in clinical outcome. The subject can be monitored in one or more of the following periods: prior to beginning of treatment; during the treatment; or after one or more elements of the treatment have been administered. Monitoring can be used to evaluate the need for further treatment with the same PSMA binding agent or for additional treatment with additional agents. Generally, a decrease in one or more of the parameters described above is indicative of the improved condition of the subject.

The methods described herein can further include methods wherein the antibody or antigen binding fragment thereof is administered in combination with one or more additional therapeutic treatment modalities, e.g., an additional treatment modality selected from the group consisting of partial or radical removal of tissue, radiation therapy, cryosurgery, phototherapy and thermotherapy. In some embodiments, the antibody or antigen binding fragment thereof is administered in combination with a cytotoxic agent selected from the group consisting of an antimetabolite, an alkylating agent, an anthracycline, or an anti-mitotic agent. In some embodiments, the antibody or antigen binding fragment thereof is administered in combination with a cytotoxic agent selected from the group consisting of taxol, cytochalasin B, vincristine, vinblastine, colchicin, tenoposide, and maytansinoid. In some embodiments, the antibody or antigen binding fragment thereof is administered in combination with a cytotoxic agent selected from the group consisting of mitomycin, etoposide, doxorubicin, adriamycin, iproplatin, epirubicin, melphalan, pacitaxel, carboplatin, altretamine, hexamethylmelamine, topotecan hydrochloride, ifosamide, daunorubicin, mitoxantrone, mithramycin, cisplatin and actinomycin D. In some embodiments, the antibody or antigen binding fragment thereof is administered in combination with a cytotoxic agent selected from the group consisting of cyclophosphamide, busulfan, 1-dehydrotestosterone, streptozotocin, dibromomannitol, glucocorticoids, procaine, tetracaine, lidocaine, propranolol, puromycin, gramicidin D, ethidium bromide, emetine, and dihydroxy anthracin dione. In some embodiments, the antibody or antigen binding fragment thereof is administered in combination with an immunomodulatory agent, e.g., an immunomodulatory agent selected from the group consisting of IL-1, IL-2, IL-4, IL-6, IL-12, interferon α, interferon γ, GM-CSF, and GCSF. Other agents that can be administered include leucovarin and mesna. The antibody or antigen binding fragment and the agent, e.g., the cytotoxic agent or immunomodulatory agent, can be formulated as pharmaceutical compositions, e.g., as a single pharmaceutical composition, or as separate pharmaceutical compositions. When the antibody or antigen binding fragment and the agent, e.g., the cytotoxic agent or immunomodulatory agent, are formulated separately, the respective pharmaceutical compositions can be mixed, e.g., just prior to administration or can be administered separately, e.g., at the same or different times.

In some embodiments, the antibody or antigen binding fragment and the agent, e.g., the cytotoxic agent or immunomodulatory agent, are administered to a subject at the same time or within an interval, such that there is overlap of an effect of each on the patient. Preferably the administration of the antibody or antigen binding fragment and the agent is spaced sufficiently close together such that an improved effect, e.g., a combinatorial effect, is achieved. The interval can be an interval of hours, days or weeks. Generally, the antibody or antigen binding fragment and the agent are concurrently bioavailable, e.g., detectable, in the subject. In a preferred embodiment at least one administration of the antibody or antigen binding fragment is made while the agent, e.g., the cytotoxic agent or immunomodulatory agent, is still present at a therapeutic level in the subject. In one embodiment the cytotoxic agent or immunomodulatory agent is administered between an earlier and a later administration of the antibody or antigen binding fragment. In other embodiments the antibody or antigen binding fragment is administered between an earlier and a later administration of the cytotoxic agent or immunomodulatory agent.

The methods of the invention can further include the step of analyzing a nucleic acid or protein from the subject, e.g., analyzing the genotype of the subject. In one embodiment, a nucleic acid encoding human PSMA and/or an upstream or downstream component(s) of human PSMA signaling, e.g., an extracellular or intracellular activator or inhibitor of human PSMA, is analyzed. The analysis can be used, e.g., to evaluate the suitability of, or to choose between alternative treatments, e.g., a particular dosage, mode of delivery, time of delivery, inclusion of adjunctive therapy, e.g., administration in combination with a second agent, or generally to determine the subject's probable drug response phenotype or genotype. The nucleic acid or protein can be analyzed at any stage of treatment, but preferably, prior to administration of the PSMA binding agent to thereby determine appropriate dosage(s) and treatment regimen(s) of the PSMA binding agent (e.g., amount per treatment or frequency of treatments) for prophylactic or therapeutic treatment of the subject.

In another aspect, the invention features methods for detecting the presence of a PSMA nucleic acid, e.g., mRNA or cDNA, or PSMA protein, in a sample, in vitro (e.g., a biological sample, such as plasma, tissue biopsy, e.g., a cancerous tissue). The subject method can be used to evaluate, e.g., diagnose or stage a disorder described herein, e.g., a cancer of the female reproductive system or a childhood cancer as described herein . The method includes: (i) contacting the sample (and optionally, a reference, e.g., a control sample) with an agent specific for a PSMA nucleic acid, e.g., a probe or a primer, or a PSMA binding agent, under conditions that allow interaction of the agent and the PSMA nucleic acid, e.g., mRNA or cDNA, or protein to occur; and (ii) detecting formation of a complex between the agent, and the sample (and optionally, a reference, e.g., a control sample). Formation of the complex is indicative of the presence of PSMA nucleic acid or protein, and can indicate the suitability or need for a treatment described herein. For example, a statistically significant change in the formation of the complex in the sample relative to the control sample is indicative of the presence of PSMA in the sample. In one embodiment, the PSMA-binding agent is an anti-PSMA antibody or antigen-binding fragment thereof, e.g., an anti-PSMA antibody or antigen-binding fragment thereof as described herein. In other embodiments, the agent is a nucleic acid that specifically hybridizes to the PSMA nucleic acid.

In yet another aspect, the invention provides a method for detecting the presence of PSMA, in vivo (e.g., in vivo imaging in a subject). The subject method can be used to evaluate, e.g., diagnose or stage a disorder described herein, e.g., a cancer of the female reproductive tract or a childhood cancer as described herein, in a subject, e.g., a mammal, e.g., a primate, e.g., a human. The method includes: (i) administering to a subject (and optionally, a reference, e.g., a control subject) a PSMA binding agent, under conditions that allow interaction of the binding agent and the PSMA protein to occur; and (ii) detecting formation of a complex between the PSMA binding agent and PSMA, e.g., a statistically significant change in the formation of the complex in the subject relative to the reference, e.g., the control subject or subject's baseline, is indicative of the presence of PSMA. In some embodiments, the presence of PSMA is indicative of the presence of a cancer of the female reproductive tract or a childhood cancer as described herein.

In other aspects, a method of diagnosing or staging a disorder described herein, e.g., a cancer of the female reproductive tract or a childhood cancer as described herein, is provided.

In some embodiments, the method includes: (i) optionally identifying a subject having, or at risk of having, a cancer described herein, e.g., a cancer of the female reproductive tract or a childhood cancer as described herein; (ii) providing a sample, e.g., a sample of a bodily fluid, tissue or cell from the subject, e.g., from a tissue that is or is suspected to be or could be affected with the disorder; (iii) contacting said sample and/or a control sample with a labeled agent specific for a PSMA nucleic acid, e.g., a probe or a primer, or a labeled PSMA binding agent, under conditions that allow interaction of the binding agent and the PSMA nucleic acid, e.g., cDNA, mRNA, or PSMA protein to occur, and (iv) detecting formation of a PSMA-binding agent complex. A statistically significant increase in the formation of the complex between the labeled agent with respect to a control sample is indicative of the presence of a cancer, e.g., a cancer described herein, e.g., a cancer of the female reproductive tract or a childhood cancer as described herein, or the stage of a cancer of the female reproductive tract or a childhood cancer as described herein.

In some embodiments, the invention provides methods for diagnosing a subject with a cancer selected from the group consisting of a cancer of the female reproductive tract, a childhood cancer or cancer affecting adults and children. The method includes optionally identifying a subject having, or at risk of having, a cancer described herein, e.g., a cancer of the female reproductive tract or a childhood cancer as described herein; providing a sample from the subject; contacting the sample with a PSMA binding agent, e.g., an antibody or antigen binding fragment thereof that binds PSMA, under conditions that allow interaction of the antibody or antigen binding fragment and PSMA to occur; and detecting the formation of a complex of PSMA and the agent, e.g., the antibody or antigen-binding fragment. The formation of agent-PSMA complexes indicates the presence of a cancer described herein, e.g., a cancer selected from the group consisting of a cancer of the female reproductive tract and a childhood cancer.

In some embodiments, the sample is a bodily fluid, e.g., serum or urine. In some embodiments, the sample is a tissue biopsy sample, e.g., a biopsy sample taken from the tissue of a tumor. Examples of tissue samples include ovary tissue for ovarian cancer, kidney tissue for Wilm's tumors and neural tissue for neuroblastomas. In some embodiments, the formation of agent-PSMA complexes is detected in the vasculature of the tissue. In some embodiments, the sample is a tissue biopsy sample and the formation of the complex can be detected in the vasculature of the tissue.

The methods of the invention also include a method of in vivo imaging of cancers having PSMA expressed in the neovasculature. The in vivo imaging can be of a cancer of the female reproductive tract a childhood cancer, or other cancer described herein. The method includes administering to the subject a PSMA binding agent, e.g., an antibody or antigen binding fragment thereof that binds to the extracellular domain of PSMA, and is detectably labeled; and detecting the formation of a complex of PSMA and the agent, e.g., the antibody or antigen binding fragment thereof, in the body of the subject. The formation of a complex is indicative of a cancer of the female reproductive tract, a childhood cancer, or other cancer described herein. In some embodiments, detecting the formation of a complex in the body of the subject provides an indication of the location of the cancerous tissue in the body of the subject. In some embodiments, detecting the formation of agent-PSMA complexes in the body of the subject provides an indication of the severity of the cancer. In some embodiments, detecting the formation of agent-PSMA complexes in the body of the subject provides an indication that the cancer is metastatic. In some embodiments, the formation of complexes is detected in the vasculature of the cancerous tissue.

Typically, the agent, e.g., the PSMA binding agent, e.g., the anti-PSMA antibody or fragment thereof, is directly or indirectly labeled with a detectable substance to facilitate detection of the bound or unbound binding agent. Suitable detectable substances include various biologically active enzymes, prosthetic groups, fluorescent materials, luminescent materials, paramagnetic (e.g., nuclear magnetic resonance active) materials, chromophores, and radioactive materials. In some embodiments, the modified anti-PSMA antibody or fragment thereof is coupled to a compound that emits radiation, e.g., a radioactive isotope, e.g., an α-emitter, a β-emitter, a γ-emitter, or a β- and γ-emitter. In some embodiments, the compound that emits radiation is selected from the group consisting of yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), rhodium (¹⁸⁸Rh), iodine (¹³¹I or ¹²⁵I), indium (¹¹¹In), technetium (⁹⁹mTc), phosphorus (³²P), carbon (¹⁴C), sulfur (³⁵S), and tritium (³H). In some embodiments, the compound that emits radiation is lutetium (¹⁷⁷Lu). In some embodiments, the compound that emits radiation is indium (¹¹¹In). In some embodiments, the formation of PSMA-binding agent complexes is detected by detecting the labeled antibody or antigen binding fragment thereof.

As used herein, “PSMA” or “prostate-specific membrane antigen” protein refers to mammalian PSMA, preferably human PSMA protein. Human PSMA includes the two protein products, PSMA and PSM′, encoded by the two alternatively spliced mRNA variants (containing about 2,653 and 2,387 nucleotides, respectively) of the PSMA cDNA disclosed in Israeli et al. (1993) Cancer Res. 53:227-230; Su et al. (1995) Cancer Res. 55:1441-1443; U.S. Pat. No. 5,538,866, U.S. Pat. No. 5,935,818, and WO 97/35616, the contents of which are hereby incorporated by reference. It is generally present as a dimer. The long transcript of PSMA encodes a protein product of about 100-120 kDa molecular weight characterized as a type II transmembrane receptor having sequence identity with the transferrin receptor and having NAALADase activity (Carter et al. (1996) Proc. Natl. Acad. Sci. USA 93:749-753). Accordingly, the term “human PSMA” refers to at least two protein products, human PSMA and PSM′, which have or are homologous to (e.g., at least about 85%, 90%, 95% identical to) an amino acid sequence as shown in Israeli et al. (1993) Cancer Res. 53:227-230; Su et al. (1995) Cancer Res. 55:1441-1443; U.S. Pat. No. 5,538,866, U.S. Pat. No. 5,935,818, and WO 97/35616; or which is encoded by (a) a naturally occurring human PSMA nucleic acid sequence (e.g., Israeli et al. (1993) Cancer Res. 53:227-230 or U.S. Pat. No. 5,538,866); (b) a nucleic acid sequence degenerate to a naturally occurring human PSMA sequence; (c) a nucleic acid sequence homologous to (e.g., at least about 85%, 90%, 95% identical to) the naturally occurring human PSMA nucleic acid sequence; or (d) a nucleic acid sequence that hybridizes to one of the foregoing nucleic acid sequences under stringent conditions, e.g., highly stringent conditions; or dimers thereof.

A “PSMA binding agent” is an agent which interacts with (e.g., binds to) PSMA, preferably human PSMA. Preferably, the PSMA binding agent interacts with, e.g., binds to, the extracellular domain of PSMA, e.g., the extracellular domain of human PSMA located at about amino acids 44-750 of human PSMA (amino acid residues correspond to the human PSMA sequence disclosed in U.S. Pat. No. 5,538,866). In some embodiments, the PSMA binding agent binds to a dimer of PSMA, e.g., the agent binds to a portion of PSMA exposed in both a dimer of PSMA and a monomer of PSMA, or the agent binds to a portion of PSMA exposed on a PSMA dimer but not a PSMA monomer. Preferably, the interaction, e.g., binding, occurs with high affinity (e.g., affinity constant of at least 10⁷ M⁻¹, preferably, between 10⁸ M⁻¹ and 10¹⁰ M⁻¹, or about 10⁹ M⁻¹) and specificity. Preferably, the PSMA binding agent treats, e.g., ablates or kills, a cell, e.g., a PSMA-expressing cell (e.g., a cancerous cell or a vascular endothelial cell). The mechanism by which the PSMA binding agent treats, e.g., ablates or kills, the cell is not critical to the practice of the invention. In some embodiments, the PSMA binding agent may bind to and be internalized with the PSMA expressed in the cells and/or vascular endothelial cells proximate to the cells. In those embodiments, the binding agent can be used to target a second moiety, e.g., a cytotoxic agent, to the cell. In other embodiments, the PSMA binding agent may mediate host mediated-killing, e.g., complement- or ADCC-mediated killing, of the cell and/or the vascular cell proximate thereto, upon binding to the extracellular domain of PSMA. The cell can be killed directly by the PSMA binding agent binding directly to the cell (e.g., to a cancerous cell) or to vascular endothelial cells proximate thereto. Alternatively, the PSMA binding agent can treat, e.g., kill or ablate, or otherwise change the properties of the vascular endothelial cells to which it binds so that blood flow to the cells proximate thereto is reduced, thereby causing the proximate cells to be killed or ablated. Examples of PSMA binding agents include anti-PSMA antibodies (e.g., monospecific, monoclonal (e.g., human or rodent), recombinant or modified, e.g., chimeric, CDR-grafted, humanized, deimmunized, in vitro generated antibodies); small molecules and peptidomimetics.

An “anti-PSMA antibody” is an antibody that interacts with (e.g., binds to) PSMA, preferably human PSMA protein. The antibody can be any PSMA-specific antibody (e.g., a monospecific, or a recombinant or modified antibody), and includes antigen-binding fragments thereof.

As used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). Preferably, each VH and VL is composed of three CDRs and four FRs, arranged from amino-terminus to carboxy-terminus in the following order: FR1, CDR1, FR2, CDR2, FR3, CDR3, FR4.

The VH or VL chain of the antibody can further include all or part of a heavy or light chain constant region. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system. The term “antibody” includes intact immunoglobulins of types IgA, IgG, IgE, IgD, IgM (as well as subtypes thereof), wherein the light chains of the immunoglobulin may be of types kappa or lambda.

As used herein, the term “immunoglobulin” refers to a protein consisting of one or more polypeptides substantially encoded by immunoglobulin genes. Recognized human immunoglobulin genes include the kappa, lambda, alpha (IgA1 and IgA2), gamma (IgG1, IgG2, IgG3, IgG4), delta, epsilon and mu constant region genes, as well as a myriad of immunoglobulin variable region genes. Full-length immunoglobulin “light chains” (about 25 Kd or 214 amino acids) are encoded by a variable region gene at the NH2-terminus (about 110 amino acids) and a kappa or lambda constant region gene at the COOH-terminus. Full-length immunoglobulin “heavy chains” (about 50 Kd or 446 amino acids), are similarly encoded by a variable region gene (about 116 amino acids) and one of the other aforementioned constant region genes, e.g., gamma (encoding about 330 amino acids). The term “immunoglobulin” includes an immunoglobulin having: CDRs from a non-human source, e.g., from a non-human antibody, e.g., from a mouse immunoglobulin or another non-human immunoglobulin, from a consensus sequence, or from a sequence generated by phage display, or any other method of generating diversity; and having a framework that is less antigenic in a human than a non-human framework, e.g., in the case of CDRs from a non-human immunoglobulin, less antigenic than the non-human framework from which the non-human CDRs were taken. The framework of the immunoglobulin can be human, humanized non-human, e.g., murine; framework modified to decrease antigenicity in humans', or a synthetic framework, e.g., a consensus sequence. These are sometimes referred to herein as modified immunoglobulins. A modified antibody, or antigen binding fragment thereof, includes at least one, two, three or four modified immunoglobulin chains, e.g., at least one or two modified immunoglobulin light and/or at least one or two modified heavy chains. In one embodiment, the modified antibody is a tetramer of two modified heavy immunoglobulin chains and two modified light immunoglobulin chains.

As used herein, “isotype” refers to the antibody class (e.g., IgM or IgG1) that is encoded by heavy chain constant region genes.

The term “antigen-binding fragment” of an antibody (or simply “antibody portion,” or “fragment”), as used herein, refers to a portion of an antibody which specifically binds to PSMA (e.g., human PSMA), e.g., a molecule in which one or more immunoglobulin chains is not full length but which specifically binds to PSMA (e.g., human PSMA protein). Examples of binding fragments encompassed within the term “antigen-binding fragment” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR) having sufficient framework to specifically bind, e.g., an antigen binding portion of a variable region. An antigen binding portion of a light chain variable region and an antigen binding portion of a heavy chain variable region, e.g., the two domains of the Fv fragment, VL and VH, can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using techniques known to those with skill in the art, and the fragments are screened for utility in the same manner as are intact antibodies.

The term “monospecific antibody” refers to an antibody that displays a single binding specificity for a particular target, e.g., epitope. This term includes a “monoclonal antibody” or “monoclonal antibody composition,” which as used herein refer to a preparation of antibodies or fragments thereof of single molecular composition.

The term “recombinant” antibody, as used herein, refers to antibodies that are prepared, expressed, created or isolated by recombinant means, such as antibodies expressed using a recombinant expression vector transfected into a host cell, antibodies isolated from a recombinant, combinatorial antibody library, antibodies isolated from an animal (e.g., a mouse) that is transgenic for human immunoglobulin genes or antibodies prepared, expressed, created or isolated by any other means that involves splicing of human immunoglobulin gene sequences to other DNA sequences. Such recombinant antibodies include humanized, CDR grafted, chimeric, deimmunized, in vitro generated (e.g., by phage display) antibodies, and can optionally include constant regions derived from human germline immunoglobulin sequences.

The methods described herein can be practiced on any subject, e.g., a mammal, (e.g., a higher primate, including on humans). As used herein, the term “subject” is intended to include human and non-human animals. Preferred human subjects include a human patient having a cancer, e.g., a cancer of the female reproductive tract a childhood cancer, or other cancer, as described herein. The term “non-human animals” of the invention includes all vertebrates, e.g., mammals, such as non-human primates (particularly higher primates), sheep, dog, rodents (e.g., mouse or rat), guinea pigs, goats, pigs, cats, rabbits, cows, and non-mammals, such as chickens, amphibians, reptiles, etc.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION OF THE INVENTION

In healthy human individuals, PSMA expression is essentially limited to low levels in the adult prostate, with very low level expression in the small intestine and brain. Thus, in women and children, PSMA is typically not expressed at significant levels, and no detectable expression is seen in normal vasculature. Therefore, PSMA expression in the neovasculature of cancers of the female reproductive system and of childhood cancer can be used to diagnose and treat these cancers. In addition, other cancers affecting adults or children can be diagnosed and treated based upon PSMA expression in the neovasculature of these cancers. Various PSMA binding agents such as antibodies, or antigen-binding fragments thereof, that are specific for PSMA, e.g., to the extracellular domain of PSMA, can be used to diagnose and to treat these cancers.

Anti-PSMA Antibodies

An anti-PSMA antibody suitable for use in the methods described herein is an antibody that interacts with (e.g., binds to) PSMA, preferably human PSMA protein. In some embodiments, the antibody interacts with a PSMA dimer, e.g., the antibody interacts with an epitope that is at least partially exposed by a PSMA homodimer, or a dimer specific epitope. A “dimer specific epitope” refers to an epitope exposed by a PSMA dimer but not by a PSMA monomer. The antibodies or fragments thereof can bind to the surface of cells expressing PSMA.

PSMA is normally recycled from the cell membrane into the cell. Thus, the antibody can be internalized with PSMA through the process of PSMA recirculation.

The antibody can be any PSMA-specific antibody (e.g., a monospecific, or a recombinant or modified antibody), and includes antigen-binding fragments thereof (e.g., Fab, F(ab′)₂, Fv or single chain Fv fragments). These include monoclonal antibodies, recombinant antibodies, chimeric antibodies, humanized antibodies, deimmunized antibodies, and human antibodies, as well as antigen-binding fragments of the foregoing. The antibodies can be of the various isotypes, including: IgG (e.g., IgG1, IgG2, IgG3, IgG4), IgM, IgA1, IgA2, IgD, or IgE. Preferably, the antibody is an IgG isotype, e.g., IgG1. In some embodiments, the modified antibodies are those having one or more complementarity-determining regions (CDRs) from a J591, J415, J533 or E99 antibody. In other embodiments, the modified antibody has one or more CDRs from an anti-PSMA antibody described, e.g., in PCT Publication No.: WO 03/064606, U.S. Patent Application Publication No. 2003034903, Schülke et al., (2003) PNAS USA, 100(27):12590-12595; Graver et al., (1998) Cancer Res. 58:4787-4789. Typically, the anti-PSMA antibody interacts with, e.g., binds to, the extracellular domain of PSMA, e.g., the extracellular domain of human PSMA located at about amino acids 44-750 of human PSMA (amino acid residues correspond to the human PSMA sequence disclosed in U.S. Pat. No. 5,538,866).

In some embodiments, the anti-PSMA antibody binds all or part of the epitope of an antibody described in U.S. Pat. Nos. 6,150,508, 6,107,090 and 6,136,311, PCT Publication No. WO 97/35616, PCT Publication No. WO 01/09192, and PCT Publication No. WO 02/098897 (the contents of which are incorporated herein by reference), e.g., one or more of J591, E99, J415, J533 or fragments thereof. In other embodiments, the anti-PSMA antibody binds all or part of an epitope recognized by an antibody described in PCT Publication No.: WO 03/064606, U.S. Patent Application Publication No. 2003034903, Schülke et al., (2003) PNAS USA, 100(27):12590-12595; Graver et al., (1998) Cancer Res. 58:4787-4789 (the contents of which are incorporated herein by reference), e.g., one or more of 4A3, 7F12, 8A11, 8C12, 16F9, 026, PSMA 4.40, PSMA 3.7, PSMA 3.8, PSMA 3.9, PSMA 3.11 PSMA 5.4, PSMA 7.3, PSMA 10.3, PSMA 1.8.3, PSMA A3.1.3, PSMA A3. 3.1, Abgenix 4.248.2, Abgenix 4.360.3, Abgenix 4.7.1, Abgenix 4.4.1, Abgenix 4.177.3, Abgenix 4.16.1, Abgenix 30 4.22.3, Abgenix 4.28.3, Abgenix 4.40.2, Abgenix 4.48.3, Abgenix 4.49.1, Abgenix 4.209.3, Abgenix 4.219.3, Abgenix 4.288.1, Abgenix 4.333.1, Abgenix 4.54.1, Abgenix 4.153. 1, Abgenix 4.232.3, Abgenix 4.292.3, Abgenix 4.304.1, Abgenix 4.78.1, Abgenix 4.152.1, or fragments thereof.

In some embodiments, the anti-PSMA antibody can inhibit, e.g., competitively inhibit, the binding of an anti-PSMA antibody such as J591, E99, J415, and J533, to human PSMA. In other embodiments, the anti-PSMA antibody can inhibit, e.g., competitively inhibit, the binding to human PSMA of an anti-PSMA antibody such as those described in U.S. Pat. No. 6,150,508, PCT Publication No. WO 01/09192, U.S. Patent Application Publication No. 2003034903, Schüke et al., PNAS USA, 100(27):12590-12595; Graver et al., (1998) Cancer Res. 58:4787-4789, e.g., one or more of 4A3, 7F12, 8A11, 8C12, 16F9, 026, PSMA 4.40, PSMA 3.7, PSMA 3.8, PSMA 3.9, PSMA 3.11 PSMA 5.4, PSMA 7.3, PSMA 10.3, PSMA 1.8.3, PSMA A3.1.3, PSMA A3. 3.1, Abgenix 4.248.2, Abgenix 4.360.3, Abgenix 4.7.1, Abgenix 4.4.1, Abgenix 4.177.3, Abgenix 4.16.1, Abgenix 30 4.22.3, Abgenix 4.28.3, Abgenix 4.40.2, Abgenix 4.48.3, Abgenix 4.49.1, Abgenix 4.209.3, Abgenix 4.219.3, Abgenix 4.288.1, Abgenix 4.333.1, Abgenix 4.54.1, Abgenix 4.153. 1, Abgenix 4.232.3, Abgenix 4.292.3, Abgenix 4.304.1, Abgenix 4.78.1, Abgenix 4.152.1, or fragments thereof. An anti-PSMA antibody can bind to an epitope, e.g., a conformational or a linear epitope, which epitope when bound prevents binding of an anti-PSMA antibody, e.g., an anti-PSMA antibody described herein such as J591, E99, J415, and J533. The epitope can be in close proximity, spatially or functionally-associated, e.g., an overlapping or adjacent epitope in linear sequence or conformationally to the one recognized by an anti-PSMA antibody described herein such as the J591, E99, J415, or J533 antibody. In some embodiments, the anti-PSMA antibody binds to an epitope located wholly or partially within the region of about amino acids 120 to 500, preferably 130 to 450, more preferably, 134 to 437, or 153 to 347, of human PSMA (amino acid residues correspond to the human PSMA sequence disclosed in U.S. Pat. No. 5,538,866). Typically, the epitope includes at least one glycosylation site, e.g., at least one N-linked glycosylation site (e.g., an asparagine residue located at about amino acids 190-200, e.g., at about amino acid 195, of human PSMA; amino acid residues correspond to the human PSMA sequence disclosed in U.S. Pat. No. 5,538,866). In some embodiments, the antibodies (or fragments thereof) are a recombinant or modified anti-PSMA antibody chosen from, e.g., a chimeric, a humanized, a deimmunized, or an in vitro generated antibody such as those described in PCT Publication No: WO 02/098897. In other embodiments, the antibodies (or fragments thereof) are human anti-PSMA antibodies such as those described in PCT Publication No.: WO 01/09192 and WO 03/064046.

Additional antibodies to PSMA can be generated using techniques known in the art. See generally, Harlow, E. and Lane, D. (1988) Antibodies: A Laboratory Manual, Cold Spring Harbor Laboratory Press, Cold Spring Harbor, N.Y. For example, monoclonal antibodies can be produced by a variety of techniques, including conventional monoclonal antibody methodology e.g., the standard somatic cell hybridization technique of Kohler and Milstein, Nature 256: 495 (1975). Although somatic cell hybridization procedures are typically used, other techniques for producing monoclonal antibodies can be employed, e.g., viral or oncogenic transformation of B lymphocytes. The typical animal system for preparing hybridomas is the murine system; hybridoma production in the mouse is an established procedure. Immunization protocols and techniques for isolation of immunized splenocytes for fusion are known in the art. Fusion partners (e.g., murine myeloma cells) and fusion procedures are also known.

Useful immunogens for the purpose of this invention include PSMA (e.g., human PSMA)-bearing cells (e.g., a prostate tumor cell line, e.g., LNCap cells, or fresh or frozen prostate tumor cells); membrane fractions of PSMA-expressing cells (e.g., a prostate tumor cell line, e.g., LNCap cells, or fresh or frozen prostate tumor cells); isolated or purified PSMA, e.g., human PSMA protein (e.g., biochemically isolated PSMA, or a portion thereof, e.g., the extracellular domain of PSMA). Techniques for generating antibodies to PSMA are described in U.S. Pat. No. 6,107,090, U.S. Pat. No. 6,136,311, U.S. Pat. No. 6,150,508, and PCT Publication No: WO 02/098897, the contents of all of which are expressly incorporated by reference. Preferably, the immunogen is dimeric.

An anti-PSMA antibody or antigen-binding fragment thereof can be functionally linked, e.g., by chemical coupling, genetic fusion, non-covalent association or otherwise, to another molecular entity, typically a detectable label or a therapeutic (e.g., a cytotoxic or cytostatic) agent or moiety. In some embodiments, the antibody or fragment thereof can be linked to another molecular entity by, e.g., a cleavable linker, e.g., a cleavable linker that allows the release of the molecular entity into the intracellular space upon internalization of the antibody-molecular entity complex.

Useful detectable agents (e.g., labels) with which an antibody or antibody portion of the invention may be derivatized (or labeled) to include fluorescent compounds, various enzymes, prosthetic groups, luminescent materials, bioluminescent materials, fluorescent emitting metal atoms, e.g., europium (Eu), and other lanthanides, and radioactive materials (described below). Exemplary fluorescent detectable agents include fluorescein, fluorescein isothiocyanate, rhodamine, 5-dimethylamine-1-napthalenesulfonyl chloride, phycoerythrin and the like. An antibody may also be derivatized with detectable enzymes, such as alkaline phosphatase, horseradish peroxidase, b-galactosidase, acetylcholinesterase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody may also be derivatized with a prosthetic group (e.g., streptavidin/biotin or avidin/biotin). For example, an antibody may be derivatized with biotin, and detected through indirect measurement of avidin or streptavidin binding. Examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of bioluminescent materials include luciferase, luciferin, and aequorin.

Labeled antibodies can be used, for example, diagnostically and/or experimentally in a number of contexts, including (i) to isolate a predetermined antigen by standard techniques, such as affinity chromatography or immunoprecipitation; (ii) to detect a predetermined antigen (e.g., in a cellular lysate or cell supernatant) in order to evaluate the abundance and pattern of expression of the protein; (iii) to monitor protein levels in tissue as part of a clinical testing procedure, e.g., to determine the efficacy of a given treatment regimen.

Radioactive isotopes can be used in diagnostic or therapeutic applications. Radioactive isotopes that can be coupled to the anti-PSMA antibodies include, but are not limited to, α-, β-, or γ-emitters, or β- and γ-emitters. Such radioactive isotopes include, but are not limited to, iodine (¹³¹I or ¹²⁵I), yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), indium (¹¹¹In), technetium (⁹⁹mTc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), or gallium (⁶⁷Ga). Radioisotopes useful as therapeutic agents include yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), and rhodium (¹⁸⁸Rh). Radioisotopes useful as labels, e.g., for use in diagnostics, include iodine (¹³¹I or ¹²⁵I), indium (¹¹¹In), technetium (⁹⁹mTc), phosphorus (³²P), carbon (¹⁴C), and tritium (³H), or one or more of the therapeutic isotopes listed above.

Examples of other therapeutic agents that can be coupled to the anti-PSMA antibodies include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020), CC-1065 (see U.S. Pat. Nos. 5,475,092, 5,585,499, 5,846,545) and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids, e.g., DM1 or maytansinol). The maytansinoid can be, for example, maytansinol or a maytansinol analogue. Examples of maytansinol analogues include those having a modified aromatic ring. Examples of maytansinol analogues include those having a modified aromatic ring (e.g., C-19-decloro, C-20-demethoxy, C-20-acyloxy) and those having modifications at other positions (e.g., C-9-CH, C-14-alkoxymethyl, C-14-hydroxymethyl or aceloxymethyl, C-15-hydroxy/acyloxy, C-15-methoxy, C-18-N-demethyl, 4,5-deoxy). Maytansinol and maytansinol analogues are described, for example, in U.S. Pat. No. 6,333,410, the contents of which are incorporated herein by reference. The calicheamicin can be, for example, a bromo-complex calicheamicin (e.g., an alpha, beta or gamma bromo-complex), an iodo-complex calicheamicin (e.g., an alpha, beta or gamma iodo-complex), or analogs and mimics thereof. Bromo-complex calicheamicins include α₁-BR, α₂-BR, α₃-BR, α₄-BR, β₁-BR, β₂-BR and γ₁-BR. Iodo-complex calicheamicins include α₁-I, α₂-I, α₃-I, β₁-I, β₂-I, δ₁-I and γ₁-BR. Calicheamicin and mutants, analogs and mimics thereof are described, for example, in U.S. Pat. Nos. 4,970,198, issued Nov. 13, 1990, 5,264,586, issued Nov. 23, 1993, 5,550,246, issued Aug. 27, 1996, 5,712,374, issued Jan. 27, 1998, and 5,714,586, issued Feb. 3, 1998, the contents of which are incorporated herein by reference. Maytansinol can be coupled to antibodies using, e.g., an N-succinimidyl 3-(2-pyridyldithio)proprionate (also known as N-succinimidyl 4-(2-pyridyldithio)pentanoate or SPP), 4-succinimidyl-oxycarbonyl-a-(2-pyridyldithio)-toluene (SMPT), N-succinimidyl-3-(2-pyridyldithio)butyrate (SDPB), 2-iminothiolane, or S-acetylsuccinic anhydride.

The therapeutic agent is not to be construed as limited to classical chemical therapeutic agents. For example, the therapeutic agent can be a protein or polypeptide possessing a desired biological activity. Such proteins can include, for example, a toxin such as abrin, ricin A, pseudomonas exotoxin, diphtheria toxin, or a component thereof (e.g., a component of pseudomonas exotoxin is PE38); a protein such as tumor necrosis factor, interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors. Similarly, the therapeutic agent can be a viral particle, e.g., a recombinant viral particle, that is conjugated (e.g., via a chemical linker) or fused (e.g., via a viral coat protein) to an anti-PSMA antibody of the invention. Introduction of the viral nucleic acid molecules, e.g., recombinant viral nucleic acid molecules, into cells that express PSMA, e.g., vascular endothelial cells associated with tumors, can occur following binding and endocytosis of the anti-PSMA antibody/viral particle conjugate or fusion.

In some embodiments, the anti-PSMA antibody is in a composition, e.g., a pharmaceutically acceptable composition, formulated together with a pharmaceutically acceptable carrier. As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g., by injection or infusion). For example, suitable pharmaceutical compositions are described in U.S. Pat. Nos. 6,150,508, 6,107,090 and 6,136,311, PCT Publication WO 97/35616, PCT Publication No: WO 02/098897, PCT Publication No. 01/09192, and in pending U.S. patent application Ser. Nos. 10/160,505, 10/379,838, and 10/449,379 (the contents of all of which are incorporated herein by reference).

The anti-PSMA antibody compositions described herein can be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Typical compositions are in the form of injectable or infusible solutions. The typical mode of administration is parenteral (e.g., intravenous, subcutaneous, intraperitoneal, intramuscular). In some embodiments, the antibody is administered by intravenous infusion or injection. In other embodiments, the antibody is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Cancer Diagnosis

The methods described herein include methods for the diagnosis of childhood cancers and cancers of the female reproductive system. The methods described herein can also be used to diagnose renal cancer (e.g., a clear cell cancer, chromophilic cancer, chromophobic cancer, oncocytic cancer or collecting duct or Bellini duct cancer), breast cancer (e.g., infiltrating ductal breast cancer (e.g., mucinous, medullary, papillary or tubular), infiltrating labular carcinoma or sarcoma) and lung cancer (e.g., small cell lung cancer, non-small cell lung cancer or bronchial gland cancer).

The invention features methods for diagnosing cancer by detecting the presence of a PSMA protein in a sample in vitro (e.g., a biological sample, e.g., serum or urine, or a tissue biopsy, e.g., from a cancerous lesion). The methods include: (i) contacting the sample (and optionally, contacting a reference, e.g., a control sample) with an anti-PSMA antibody as described herein, under conditions that allow interaction of the anti-PSMA antibody and the PSMA protein to occur; and (ii) detecting formation of any complexes between the anti-PSMA antibody, and the sample (and optionally, the reference, e.g., control, sample). Formation of a complex is indicative of the presence of PSMA protein, and can indicate the presence of a neoplastic growth, e.g., a growth associated with a cancer of the female reproductive tract, a childhood cancer, or other cancer, as described herein. For example, a statistically significant change in the formation of the complex in the sample relative to the reference sample, e.g., the control sample, is indicative of the presence of PSMA in the sample. In some embodiments, the methods can include the use of more than one anti-PSMA antibody, e.g., two anti-PSMA antibodies that bind to different epitopes on PSMA. In some embodiments, the method involves an ELISA assay. In some embodiments, the anti-PSMA antibody is a modified antibody, e.g., labeled, e.g., radiolabeled or labeled with a fluorochrome, as described herein, to allow for direct detection of the antibody, e.g., using in vivo imaging methods.

In some embodiments, the method can be used to select a subject for administration of a composition as described herein, e.g., a composition comprising an anti-PSMA antibody (or fragment thereof), e.g., coupled to a therapeutic agent, to treat the subject. For example, if the presence of PSMA is detected in a sample derived from a subject, that subject can then be selected for administration of an anti-PSMA antibody, e.g., an antibody described herein, e.g., a modified anti-PSMA antibody.

In yet another aspect, the invention provides a method for detecting the presence of PSMA in vivo (e.g., in vivo imaging in a subject). The method can be used to evaluate, diagnose, or monitor a cancerous disorder, e.g., a childhood cancer, a cancer of the female reproductive system or other cancer described herein, in a subject, e.g., a mammal, e.g., a primate, e.g., a human. The method includes: (i) administering to a subject an anti-PSMA antibody (e.g., a modified, e.g., labeled) anti-PSMA antibody as described herein), under conditions that allow interaction of the anti-PSMA antibody and the PSMA protein to occur; and (ii) detecting formation of any complexes between the antibody or fragment thereof and PSMA. A statistically significant change in the formation of complexes in the subject relative to a reference, e.g., a control subject or the same subject's baseline, is indicative of the presence of the PSMA, and thus can be indicative of the presence, location and severity (e.g., tumor size, and whether the cancer is localized or metastasized) of cancerous tissue. In some embodiments, the method can be used to select a subject for administration of a composition as described herein, e.g., a composition comprising an anti-PSMA antibody (or fragment thereof), e.g., an anti-PSMA antibody described herein, coupled to a therapeutic agent, to treat the subject. For example, if the presence of PSMA is detected in a sample derived from a subject, that subject can then be selected for administration of an anti-PSMA antibody (e.g., a modified anti-PSMA antibody as described in PCT Publication No. WO 02/098897, and in pending U.S. patent application Ser. Nos. 10/160,505, 10/379,838, and 10/449,379).

A number of methods are known for the detection of the formation of antibody-PSMA complexes in vivo. Typically, the anti-PSMA antibody will be labeled. In the case of a radiolabeled antibody, the antibody is administered to the patient, localizes to the tumor bearing the antigen with which the antibody reacts, and is detected or “imaged” in vivo using known techniques such as radionuclear scanning using e.g., a gamma camera or emission tomography. See e.g., A. R. Bradwell et al., “Developments in Antibody Imaging,” in Monoclonal Antibodies for Cancer Detection and Therapy, R. W. Baldwin et al., (eds.), pp 65-85 (Academic Press 1985), which is hereby incorporated by reference herein. Alternatively, a positron emission transaxial tomography scanner, such as the one designated Pet VI located at Brookhaven National Laboratory, can be used where the radiolabel emits positrons (e.g., 11C, 18F, 15O, and 13N). Alternatively, fluorophore-labeled antibodies can be used, and detected using imaging methods known in the art.

Such methods can also be used to evaluate the stage of the cancer, e.g., a cancer described herein, e.g., a cancer of the female reproductive tract or childhood cancer.

Anti-Cancer Treatments

The methods can further include treating a subject, e.g., one diagnosed with a childhood cancer or a cancer of the female reproductive system, by administering a therapeutically effective amount of an anti-PSMA antibody, or antigen-binding fragment thereof. This can be administered in place of, or in addition to, an existing cancer treatment. In addition, the methods can further include treating a subject diagnosed with cancer selected from renal cancer (e.g., a clear cell cancer, chromophilic cancer, chromophobic cancer, oncocytic cancer or collecting duct or Bellini duct cancer), breast cancer (e.g., infiltrating ductal breast cancer (e.g., mucinous, medullary, papillary or tubular), infiltrating labular carcinoma or sarcoma) and lung cancer (e.g., small cell lung cancer, non-small cell lung cancer or bronchial gland cancer) by administering an anti-PSMA antibody or antigen binding fragment thereof, e.g., as described herein.

As used herein, the term “treat”, in the context of treating a disorder or subject, or “treatment”, in the context of the treatment of a disorder or a subject, is defined as the application or administration of an anti-PSMA binding agent (or other drug or other treament modality, e.g., radiation, e.g., a cancer treatment), to a subject, e.g., a patient, or the application or administration to an isolated tissue or cell from a subject, e.g., a patient, which is returned to the patient. Depending on the nature and severity of the cancer, and the risk of metastasis or recurrence, one or more treatments can be administered to the subject. The subject can be a patient having a disorder, e.g., a disorder described herein, or a symptom of a disorder. The treatment can be to cure, alleviate, palliate, improve or otherwise affect the cancer, or a symptom of the cancer. Treatment includes administering the binding agent at any stage of the disorder. It is particularly preferable to administer the binding agent at very early stages of neovascularization in a cancer. Thus, treat or treatment refers to a practice that cures, alleviates, palliates or generally lessens a disorder, or a symptom thereof, or improves an aspect of the subject's health or abilities that could be compromised or degraded by the disorder. Treatment generally begins after onset of the disorder. While not wishing to be bound by theory, treating is believed to cause the inhibition, ablation, or killing of a cell in vitro or in vivo, or otherwise reduce the capacity of a cell, e.g., an aberrant cell, to mediate a childhood cancer or a cancer of the female reproductive system as described herein.

In some embodiments, the binding agent is administered to the subject to prevent a disorder, e.g., a disorder as described herein, e.g., a childhood cancer, a cancer of the female reproductive system, or other cancer described herein. The binding agent can be administered to a subject having a predisposition to the disorder. The subject can be one at risk for the disorder, e.g., a subject having a relative afflicted with the disorder, e.g., a subject with one or more of a grandparent, parent, uncle or aunt, sibling, or child who has or had the disorder, or a subject having a genetic trait associated with risk for the disorder. In some embodiments, the disorder is a cancer of the female reproductive system, and the subject has one or more of a grandmother, mother, aunt, sister, or daughter who has or had the disorder, or a subject having a genetic trait associated with risk for the disorder. In some embodiments, the disorder is a childhood cancer, and the subject has one or more of a grandmother, grandfather, mother, father, aunt, uncle, sister, or brother who has or had the disorder, or a subject having a genetic trait associated with risk for the disorder. In preventative embodiments, the binding agent is administered prior to clinically recognized onset of the disorder.

As used herein, an amount of a PSMA binding agent, e.g., an anti-PSMA antibody, effective to treat a disorder described herein, or a “therapeutically effective amount,” refers to an amount of the agent which is effective, upon single or multiple dose administration to a subject, in treating a cell, e.g., a cancerous cell (e.g., a PSMA-expressing cancerous vascular endothelial cell), or in prolonging curing, alleviating, relieving or improving a subject with a disorder as described herein beyond that expected in the absence of such treatment. As used herein, “inhibiting the growth” of a cancer refers to slowing, interrupting, arresting or stopping its growth and does not necessarily indicate a total elimination of the growth or cancer.

As used herein, an amount of a PSMA binding agent, e.g., an anti-PSMA antibody, effective to prevent a disorder, or a “prophylactically effective amount” of the agent refers to an amount of a binding agent, e.g., an anti-PSMA antibody, e.g., an anti-PSMA antibody as described herein, which is effective, upon single- or multiple-dose administration to the subject, in preventing or delaying the occurrence of the onset or recurrence of a disorder, e.g., a childhood cancer, a cancer of the female reproductive system, or other cancer, as described herein, or treating a symptom thereof.

The terms “induce,” “inhibit,” “potentiate,” “elevate,” “increase,” “decrease” or the like, e.g., which denote quantitative differences between two states, refer to a difference, e.g., a statistically significant difference, between the two states. For example, “an amount effective to inhibit the proliferation of the PSMA-expressing hyperproliferative cells” means that the rate of growth of the treated cells will be different, e.g., statistically significantly different, from the untreated cells.

Dosage regimens can be adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions formulated in dosage unit form provide ease of administration and uniformity of dosage. Dosage unit form as used herein refers to physically discrete units suited as unitary dosages for the subjects to be treated; each unit typically contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of an antibody administered according to the methods of the invention is 0.1-20 mg/kg, more preferably 1-10 mg/kg. In one embodiment, the anti-PSMA antibody can be administered by intravenous infusion at a rate of less than 10 mg/min, preferably less than or equal to 5 mg/min to reach a dose of about 1 to 500 mg/m², preferably about 10 to 400 mg/m², about 18 to 350 mg/m², and more preferably, about 250-280 mg/m². The anti-PSMA antibody can be administered in a single dose or in multiple doses. The dosage schedule can be varied, such that the antibody is administered once, twice, three or more times per week for any number of weeks or the antibody is administered more than once (e.g., two, three, four, five, six, seven times) with administration occurring once a week, once every two, three, four, five, six, seven, eight, nine or ten weeks. In some embodiments, the anti-PSMA antibody molecule can be administered once a week for six weeks for a total of six doses, or twice a week for six weeks for a total of twelve doses.

In some embodiments, the anti-PSMA antibody molecule is conjugated to a therapeutic agent such as DM1, and can be administered in doses of about 13 to 23 mg/m² (e.g., 18 mg/m²), 27 to 37 mg/m² (e.g., 32 mg/m²), 46 to 56 mg/m² (, e.g., 51 mg/m²), or 66 to 76 mg/m² (e.g., 71 mg/m²), twice a week for six weeks. In other embodiments, the anti-PSMA antibody molecule, e.g., an antibody molecule described herein, e.g., an antibody molecule conjugated to a therapeutic agent such as DM1, can be administered in doses of about 66 to 76 mg/m² (e.g., 71 mg/m²), 87 to 97 mg/m² (e.g., 92 mg/m²), 115 to 125 mg/m² (e.g., 120 mg/m²), or 151 to 161 mg/m² (e.g., 156 mg/m²), once a week for six weeks.

In some embodiments, the methods of the invention include administering to the subject two or more doses of an antibody molecule described herein coupled to lutetium (¹⁷⁷Lu), wherein each dose is about 40 to 65%, preferably about 40% to 60%, 45% to 55% of the maximum tolerated dose (MTD) of the antibody molecule coupled to lutetium (¹⁷⁷Lu). The antibody coupled to ¹⁷⁷Lu can be given in two, three, four, five, six, seven, eight, nine or ten doses, e.g., over a period of a dose once every week, two weeks, three weeks, four weeks, five weeks, six weeks, seven weeks, eight weeks, or more. In a preferred embodiment, the subject is administered up to three, four or five doses, e.g., with a dose administered once every four to eight weeks. Each dose can be at about the same amount as the other doses or one or more doses can differ from each other so long as no dose given is greater than 65% of the MTD of the antibody molecule coupled to ¹⁷⁷Lu. In one embodiment, the method of treating or preventing a cancerous disorder as described herein includes administering to the subject two or more doses of a deimmunized J591 antibody, e.g., a deimmunized J591 as described herein, coupled to ¹⁷⁷Lu, wherein each dose is administered at less than 60 mCi/m². Preferably, each dose of the deimmunized J591 antibody molecule coupled to ¹⁷⁷Lu is administered at less than 45 mCi/m², e.g., 30 mCi/m², 15 mCi/m² or less.

It is to be further noted that dosage values may vary with the type and severity of the condition to be alleviated. In addition, for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The methods described herein include the administration of an anti-PSMA antibody. The antibody can be administered by any suitable route of administration, e.g., selected depending on the location of the cancer or suspected cancer and/or the formulation of the anti-PSMA antibody-containing composition. The anti-PSMA antibody or fragment thereof, e.g., a modified anti-PSMA antibody or fragment thereof as described herein, can be administered to the subject systemically (e.g., orally, parenterally, subcutaneously, intravenously, rectally, intravaginally, intramuscularly, intraperitoneally, intranasally, transdermally, or by inhalation or intracavitary installation), topically, or by application to mucous membranes, such as the nose, throat, vagina and bronchial tubes. Typically, the antibody will be administered parenterally.

As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. RobiNS0n, ed., Marcel Dekker, Inc., New York, 1978.

In certain embodiments, an antibody or antibody portion of the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Therapeutic compositions can be administered with medical devices known in the art.

In some embodiments, a therapeutic composition described herein can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other implants, delivery systems, and modules are known to those skilled in the art.

In some embodiments, the method includes selecting and administering more than one treatment to a subject, e.g., a subject determined to have a cancer of the female reproductive system or a childhood cancer, with neovasculature containing PSMA-expressing cells. Thus, the methods described herein include selecting combinations of anti-cancer therapies. For example, the combination therapy can include an anti-PSMA antibody coformulated with, and/or coadministered with, one or more additional therapeutic agents, e.g., one or more anti-cancer agents, cytotoxic or cytostatic agents, hormone treatment, vaccines, and/or other immunotherapies. Hormone treatment, e.g., for treating a cancer of the female reproductive system such as ovarian cancer, can include one or more of: progestin, progesterone, estrogen, androgen, and gonadotrpohin releasing hormone.

In some embodiments, the anti-cancer methods can include administration of an anti-PSMA antibody in combination with one or more other therapeutic treatment modalities, including surgery, radiation, cryosurgery, phototherapy and/or thermotherapy. In some embodiments, an anti-PSMA antibody or antigen binding portion thereof can be administered as an adjuvant prior to one or more therapeutic treatment modalities. Such combination therapies may advantageously utilize lower dosages of the administered therapeutic agents, thus avoiding possible toxicities or complications associated with the various monotherapies. In yet other embodiments, the methods can be used in combination with immunomodulatory agents, e.g., IL-1, 2, 4, 6, or 12, or interferon alpha or gamma, or immune cell growth factors such as GCSF and/or GM-CSF. Other agents include leucovarin and mesna.

Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated. The anti-PSMA antibody and other therapeutic modalities can be administered during periods of active disorder, or during periods of remission or less active disease. In some embodiments, the delivery of one treatment is still occurring when the delivery of the second begins, so that there is overlap. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The anti-PSMA antibody or antigen binding fragment thereof can be administered before, concurrently with, or after the administration of another agent, e.g., a chemotherapeutic or irradiation treatment. The subject can be administered a first course of a chemotherapeutic agent or radiation, and then, e.g., if a particular result is not attained, be administered an anti-PSMA antibody or antigen binding fragment thereof. The antibody or fragment thereof can be provided concurrently or before a second course of a chemotherapeutic or irradiation agent which can be different or the same as from the first round of treatment. Thus, in some embodiments, an anti-PSMA antibody or fragment thereof can be administered with one or more other therapeutic modalities, e.g., as a first round of chemotherapeutic treatment. In other embodiments, an anti-PSMA antibody or fragment thereof can be administered as part of a later round of treatment, e.g., in combination with one or more other therapeutic modalities administered to the subject during a previous round of treatment. In some embodiments, an anti-PSMA antibody or fragment thereof can be administered to a subject that did not achieve the desired effect in previous rounds of treatment with other therapeutic modalities. The anti-PSMA antibody or fragment thereof can be administered in conjugated or unconjugated form.

Examples of existing anti-cancer treatments which can be administered in combination with an anti-PSMA antibody in the methods described herein include, but are not limited to: surgery (e.g., radical hysterectomy, unilateral oophorectomy, bilateral oophorectomy, omentectomy, simple nephrectomy, partial nephrectomy or radical nephrectomy); radiation therapy (e.g., external-beam therapy which involves three dimensional, conformal radiation therapy where the field of radiation is designed to conform to the volume of tissue treated; interstitial-radiation therapy where seeds of radioactive compounds are implanted using ultrasound guidance; and a combination of external-beam therapy and interstitial-radiation therapy); and chemotherapy.

Examples of chemotherapeutic agents include cytochalasin B, ethidium bromide, altretamine, hexamethylmelamine, melphalan, emetine, etoposide, tenoposide, colchicine, dihydroxy anthracin dione, 1-dihydrotestosterone, glucocorticoids, procaine, tetracaine, lidocaine, and propranolol. Chemotherapeutic agents also include, but are not limited to, antimetabolites (e.g., methotrexate, 6-mercaptopurine, 6-thioguanine, cytarabine, 5-fluorouracil decarbazine), alkylating agents (e.g., mechlorethamine, iproplatin, thioepa chlorambucil, CC-1065, melphalan, carmustine (BSNU) and lomustine (CCNU), cyclophosphamide, ifosfamide, busulfan, dibromomannitol, streptozotocin, carboplatin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), mitomycin, bleomycin, epirubicin, mitoxantrone, mithramycin, puromycin, gramicidin D, and anthramycin (AMC)), and anti-mitotic agents (e.g., vincristine, vinblastine, taxol, pacitaxel, and maytansinoids, e.g., maytansinol (see U.S. Pat. No. 5,208,020), and CC-1065 (see U.S. Pat. Nos. 5,475,092, 5,585,499, 5,846,545)), topoisomerase inhibitors (e.g., topotecan hydrochloride, teniposide), intercalating agents, agents capable of interfering with signal transduction, and agents that promote apoptosis, and analogs or homologs thereof. In one embodiment, the subject has ovarian cancer and an anti-PSMA antibody or fragment thereof is administered in combination with one or more of melphalan, pacitaxel, carboplatin, altretamine, hexamethylmelamine, topotecan hydrochloride, ifosfamide, cisplatin, doxorubicin, etoposide and 5-fluorouracil. Preferably, the anti-PSMA antibody or antigen binding fragment thereof is administered in combination with one or more of pacitaxel, carboplatin and cisplatin. The anti-PSMA antibody or fragment thereof can be administered before, concurrently, or after the administration of the chemotherapeutic agent. In another embodiment, the subject has Wilm's tumor and an anti-PSMA antibody or antigen binding fragment thereof is administered in combination with one or more of: vincristine, dactinomycin, doxorubicin, cyclophosphamide, etoposide, ifosfamide and carboplatin. Preferably, the anti-PSMA antibody or antigen binding fragment thereof is administered with one or more of vincristine, dactinomycin and doxorubicin. The anti-PSMA antibody or fragment thereof can be administered before, concurrently, or after the administration of the chemotherapeutic agent. In another embodiment, the subject has neuroblastoma and an anti-PSMA antibody or antigen binding fragment thereof is administered in combination with one or more of: cyclophosphamide, cisplatin, doxorubicin, teniposide, etoposide, ifosfamide, carboplatin, iproplatin, epirubicin and vincristine. The anti-PSMA antibody or fragment thereof can be administered before, concurrently, or after the administration of the chemotherapeutic agent.

In some embodiments, the anti-PSMA antibody can be administered in combination with a second antibody that does not bind to PSMA, e.g., any antibody known in the art that is useful in treating or preventing cancers of the female reproductive system or childhood cancers. For example, a number of antibodies are known and in development for the treatment of ovarian cancers, including AR54 (AltaRx), Herceptin® (trastuzumab) (Genentech/NCI), HumaRAD (16.88/88BV59) (Intracel), HumAspect® (volumtumab) (Intracel), IMC-C225 (Erbitux™) (Imclone), MDX-210 (Immuno-Designed Molecules), OvaRex® MAb (oregovemab) (Abbott), SGN-15 (cBR96-doxorubicin immunoconjugate) (Seattle Genetics), SS1(dsFv)-PE38 (NeoPharm), Theragyn (pentumomab) (Antisoma), TriAb (Titan Pharmaceuticals). For cervical cancer, SS1(dsFv)-PE38 (NeoPharm) can be used. For neuroblastoma, CH14.18 and 3F8 (anti-G_{D2} disialoganglioside antibodies) can be used.

When an anti-PSMA antibody is selected as the treatment, the anti-PSMA antibody, e.g., a modified anti-PSMA antibody, or antigen-binding fragment thereof, e.g., as described herein or in U.S. Pat. Nos. 6,107,090, 6,136,311, and 6,150,508, and PCT Publication Nos: WO 01/09192 and WO 02/098897, U.S. Patent Application Publication No. 2003034903, PCT Publication No. WO 03/064046, Schülke et al., (2003) PNAS USA, 100(27):12590-12595, and Graver et al., (1998) Cancer Res. 58:4787-4789, e.g., can be administered to a subject, or used in vitro, in non-derivatized or unconjugated forms. In other embodiments, the anti-PSMA antibody, or antigen-binding fragment thereof, can be derivatized or linked to another molecular entity, typically a label or a therapeutic (e.g., a cytotoxic or cytostatic) agent.

The molecular entity can be, e.g., another peptide, protein (including, e.g., a viral coat protein of, e.g., a recombinant viral particle), a non-peptide chemical compound, a radioactive isotope, etc. The anti-PSMA antibody, or antigen-binding fragment thereof, can be functionally linked, e.g., by chemical coupling, genetic fusion, non-covalent association or otherwise, to one or more other molecular entities. For example, the anti-PSMA antibody, or antigen-binding fragment thereof, can be coupled to a label, such as a fluorescent label, a biologically active enzyme label, a radioisotope (e.g., a radioactive ion), a nuclear magnetic resonance active label, a luminescent label, or a chromophore.

In other embodiments, the anti-PSMA antibody, or antigen-binding fragment thereof, can be coupled to a therapeutic agent, e.g., a cytotoxic moiety, e.g., a therapeutic drug, a radioisotope, molecules of plant, fungal, or bacterial origin, or biological proteins (e.g., protein toxins) or particles (e.g., recombinant viral particles, e.g., via a viral coat protein), or mixtures thereof. The therapeutic agent can be an intracellularly active drug or other agent, such as short-range radiation emitters, including, for example, short-range, high-energy α-emitters. In some embodiments, the anti-PSMA antibody, or antigen binding fragment thereof, can be coupled to a molecule of plant or bacterial origin (or derivative thereof), e.g., a maytansinoid (e.g., maytansinol or the DM1 maytansinoid), a taxane, or a calicheamicin. A radioisotope can be an α-, β-, or γ-emitter, or an β- and γ-emitter. Radioisotopes useful as therapeutic agents include yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), and rhodium (¹⁸⁸Rh). Radioisotopes useful as labels, e.g., for use in diagnostics, include iodine (¹³¹I or ¹²⁵I), indium (¹¹¹In), technetium (⁹⁹mTc), phosphorus (³²P), carbon (¹⁴C), and tritium (³H), or one of the therapeutic isotopes listed above.

The anti-PSMA antibody, or antigen-binding fragment thereof can also be linked to another antibody to form, e.g., a bispecific or a multispecific antibody. Examples of other agents which can be used in an anti-PSMA antibody therapy are described, e.g., in U.S. Pat. Nos. 6,107,090 and 6,136,311, and PCT Publication No: WO 02/098897.

The methods described herein, e.g., methods of treatment or preventing cancer, can further include the step of monitoring the subject, e.g., for a change (e.g., an increase or decrease) in one or more of tumor size; levels of a cancer marker; the rate of appearance of new lesions, e.g., in a bone scan; the appearance of new disease-related symptoms; quality of life, e.g., amount of disease associated pain, e.g., bone pain; or any other parameter related to clinical outcome. The subject can be monitored in one or more of the following periods: prior to beginning of treatment; during the treatment; or after one or more elements of the treatment have been administered. Monitoring can be used to evaluate the need for further treatment with the same anti-PSMA antibody or fragment thereof or for additional treatment with additional agents. Generally, a decrease in one or more of the parameters described above is indicative of the improved condition of the subject.

The invention is further described in the following examples, which do not limit the scope of the invention described in the claims.

### EXAMPLES

### Example 1

### Prostate Specific Membrane Antigen (PSMA) Expression in Non-Prostate Cancers

PSMA target validation was performed on a series of fresh frozen non-prostate cancer malignancies by transcriptional profiling (TP) using cDNA microarrays on nylon membranes, RT-PCR (Taqman™), in situ hybridization, western blotting, dual co-localization immunofluorescence and immunohistochemistry both before and after laser capture microdissection (LCM).

Results: PSMA mRNA expression was localized to the neo-vasculature in 55% of a series of breast, colon, lung and ovarian cancers using in situ hybridization. PSMA mRNA expression measured by Taqman™ RT-PCR was localized to the endothelium of the tumor blood vessels after microdissection. Using immunohistochemistry with the deJ591 antibody, which binds to the external domain of PSMA, on frozen sections, 40% of the same cancers were positive for PSMA immunoreactivity of the tumor vasculature as with the in situ studies. Dual immunofluorescence studies using antibodies to PSMA (deJ591) and CD31 (PECAM-1), an endothelial cell marker, localized PSMA expression to the endothelium of neo-vasculature in carcinomas of the breast, colon, lung and ovary, in Wilm's tumors and neuroblastomas, but not in the tumor vessels of prostate cancers. Using an antibody that specifically binds to the internal region of human PSMA, namely the 7E11 antibody, on paraffin sections, PSMA staining was observed in 9/10 clear cell renal cell carcinomas, in 7/10 infiltrating ductal breast cancers, in 6/10 invasive colorectal cancers and in 4/10 non-small cell lung cancer.

Conclusion: These molecular studies confirm that PSMA expression is highly associated with the neo-vasculature of non-prostate cancers and co-localizes with endothelial cell markers. PSMA is a target for both diagnostic imaging and antibody-based therapies for non-prostate cancer.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a subject having a cancer selected from the group consisting of a cancer of the female reproductive tract and a childhood cancer, the method comprising administering to the subject an effective amount of an anti-PSMA antibody or antigen binding fragment thereof that binds to the extracellular domain of PSMA, thereby treating the cancer.

2. The method of claim 1, wherein the cancer is a cancer of the female reproductive tract.

3. The method of claim 2, wherein the cancer of the female reproductive tract is selected from the group consisting of ovarian, cervical, endometrial, uterine, vaginal, vulvar or pelvic cancers and gestational trophoblastic tumors.

4. The method of claim 2, wherein the female reproductive tract cancer is ovarian cancer.

5. The method of claim 1, wherein the cancer is a childhood cancer.

6. The method of claim 5, wherein the childhood cancer is selected from the group consisting of leukemias, neuroblastomas, brain cancers, lymphomas, Wilm's tumors, bone cancers, retinoblastomas, rhabdomyosarcomas, and ovarian germ cell tumors.

7. The method of claim 5, wherein the childhood cancer is Wilm's tumor.

8. The method of claim 5, wherein the childhood cancer is neuroblastoma.

9. The method of claim 1, wherein the antibody or antigen binding fragment thereof has a light chain variable region comprising one or more complementarity determining regions (CDRs) from a monoclonal antibody selected from the group consisting of J591, J415, J533 and E99.

10. The method of claim 1, wherein the modified anti-PSMA antibody or antigen binding fragment thereof is coupled to a cytotoxic agent.

11. The method of claim 1, wherein the cytotoxic agent is selected from the group consisting of taxol, cytochalasin B, vincristine, vinblastine, colchicin, tenoposide, and maytansinoid.

12. A method of diagnosing a subject with a cancer selected from the group consisting of a cancer of the female reproductive tract and a childhood cancer, the method comprising:
providing a sample from the subject;
contacting the sample with an antibody or antigen binding fragment thereof that binds PSMA, under conditions that allow interaction of the antibody or antigen binding fragment and PSMA to occur; and
detecting the formation of a complex of PSMA and the antibody or antigen-binding fragment,
wherein the formation of antibody-PSMA complexes indicates the presence of a cancer selected from the group consisting of a cancer of the female reproductive tract and a childhood cancer.

13. The method of claim 12, wherein the cancer is a cancer of the female reproductive tract.

14. The method of claim 13, wherein the cancer of the female reproductive tract is selected from the group consisting of ovarian, cervical, endometrial, uterine, vaginal, vulvar or pelvic cancers and gestational trophoblastic tumors.

15. The method of claim 13, wherein the cancer of the female reproductive tract is ovarian cancer.

16. The method of claim 12, wherein the cancer is a childhood cancer.

17. The method of claim 16, wherein the childhood cancer is selected from the group consisting of leukemias, neuroblastomas, brain cancers, lymphomas, Wilm's tumors, bone cancers, retinoblastomas, rhabdomyosarcomas, and ovarian germ cell tumors.

18. The method of claim 16, wherein the childhood cancer is Wilm's tumor.

19. The method of claim 16, wherein the childhood cancer is neuroblastoma.

20. The method of claim 12, wherein the sample comprises a bodily fluid.

21. The method of claim 20, wherein the bodily fluid is serum.

22. The method of claim 12, wherein the sample is a tissue biopsy sample and the formation of the complex can be detected in the vasculature of the tissue.

23. The method of claim 12, wherein the antibody or antigen binding fragment thereof is a monoclonal antibody.

24. The method of claim 12, wherein the antibody or antigen binding fragment thereof competitively inhibits binding of a monoclonal antibody selected from the group consisting of J591, J415, J533, and E99.

25. The method of claim 12, wherein the antibody or antigen binding fragment thereof is labeled.

26. A method of in vivo imaging of PSMA-expressing cancers selected from the group consisting of a cancer of the female reproductive tract and childhood cancer, the method comprising:
administering to the subject an antibody or antigen binding fragment thereof that binds to the extracellular domain of PSMA and is detectably labeled; and
detecting the formation of a complex of PSMA and the antibody or antigen binding fragment thereof in the body of the subject,
wherein the formation of a complex is indicative of a cancer of the female reproductive tract or a childhood cancer.

27. The method of claim 26, wherein the cancer is a cancer of the female reproductive tract.

28. The method of claim 27, wherein the cancer of the female reproductive tract is selected from the group consisting of ovarian, cervical, endometrial, uterine, vaginal, vulvar or pelvic cancers and gestational trophoblastic tumors.

29. The method of claim 27, wherein the cancer of the female reproductive tract is ovarian cancer.

30. The method of claim 26, wherein the cancer is a childhood cancer.

31. The method of claim 30, wherein the childhood cancer is selected from the group consisting of leukemias, neuroblastomas, brain cancers, lymphomas, Wilm's tumors, bone cancers, retinoblastomas, rhabdomyosarcomas, and ovarian germ cell tumors.

32. The method of claim 31, wherein the childhood cancer is Wilm's tumor.

33. The method of claim 31, wherein the childhood cancer is neuroblastoma.

34. The method of claim 26, wherein detecting the formation of a complex in the body of the subject provides an indication of the location of the cancerous tissue in the body of the subject.

35. The method of claim 26, wherein the formation of complexes is detected in the vasculature of the cancerous tissue.

36. The method of claim 26, wherein the antibody or antigen binding fragment thereof is a monoclonal antibody.

37. The method of claim 26, wherein the antibody or antigen binding fragment thereof has a light chain variable region comprising one or more complementarity determining regions (CDRs) from a monoclonal antibody selected from the group consisting of J591, J415, J533 and E99.
